(19) United States
(12) Patent Application Publication
(10) Pub. No.: US 2015/0192565 A1
(43) Pub. Date: Jul. 9, 2015

(54) METHOD FOR EVALUATING THERAPEUTIC EFFECT OF THALIDOMIDE AND APPLICATION THEREOF

(71) Applicant: NATIONAL TAIWAN UNIVERSITY, Taipei City (TW)

(72) Inventors: Po-Huang Lee, Taipei City (TW); Tzu-Min Hung, Taipei City (TW); Yen-Chun Liu, Taoyuan City (TW)

(21) Appl. No.: 14/591,366

(22) Filed: Jan. 7, 2015

(30) Foreign Application Priority Data
Jan. 7, 2014 (TW) 103100559

(51) Int. Cl. G01N 33/50

(52) U.S. Cl. CPC G01N 33/5011

(57) ABSTRACT

The present disclosure is directed to a method for evaluating the therapeutic effect of Thalidomide and an application thereof, which evaluates the therapeutic effect of Thalidomide on liver cancer of a patient according to the expression level of the LHX4 gene in the liver of the patient.

### CROSS-REFERENCE TO RELATED APPLICATION AND CLAIM OF PRIORITY

This application claims the benefit of Taiwan Patent Application No. 103100559, filed on Jan. 7, 2014, at the Taiwan Intellectual Property Office, the disclosure of which is incorporated herein in its entirety by reference.

### TECHNICAL FIELD

The present disclosure is directed to a method for evaluating the therapeutic effect of Thalidomide and application thereof, which evaluates the therapeutic effect of Thalidomide on liver cancer in a patient according to the expression level of the LHX4 gene in the liver of the patient.

### BACKGROUND

Hepatocellular carcinoma (HCC) is the sixth most common cancer worldwide and the third most common cause of cancer-related mortality. So far, a surgical resection of the tumor remains the most effective therapy to cure HCC. However, patients who have undergone curative (surgical) resection have a high rate of HCC recurrence, approximately 80-90% within the first five years, and HCC recurrence often results in the patient's death. Therefore, how to prevent HCC recurrence after a surgical resection is a significant unmet clinical need. Since 1994, several approaches such as postoperative transcatheter arterial chemoembolization (TACE), chemotherapy and interferon alpha (IFN-α) therapy have been reported to decrease the HCC recurrence rate as an adjuvant therapy. In addition, Thalidomide has been indicated that it may serve as a promising choice to decrease the HCC recurrence rate.

Thalidomide was approved in the 1950s in Europe and clinically used as a sedative or an anti-inflammatory agent. However, due to its teratogenic effect on a developing fetus, Thalidomide was prohibited for use shortly after being sold. In 1994, Thalidomide was proven to have anti-angiogenesis activity and was considered as a potential agent for tumors such as melanoma and Kaposi sarcoma.

Similar to other solid tumors, HCC is very vascular and dependent on angiogenesis for tumor progression and metastatic proliferation. As a result, Thalidomide, with anti-angiogenesis activity, served as a potential approach for treating advanced HCC. A study shows that, for adjuvant therapy after surgical resection, the two-year HCC recurrence-free survival rate in patients receiving Thalidomide was 65%, while in patients receiving a placebo, it was 33%, which indicates the effectiveness and potential of Thalidomide as an adjuvant therapy to HCC. However, although recent findings suggest many kinds of adjuvant therapy to prevent tumor recurrence and prolong survival, the responses were often far from satisfactory due to the limited evidence to identify the specific patient population which is most likely to benefit from these adjuvant therapies.

Lhx4 protein, a LIM-homeodomain (LIM-HD) family transcription factor, contains a cysteine-rich zinc-binding domain, and participates in pituitary development and differentiation.

U.S. patent application No. 2003/0092009 discloses that the LHX4 gene is a neoplastic molecular marker for the detection of cancer in humans. U.S. patent application No. 2012/0004855 discloses that the methylation status of locus of the LHX4 gene is indicative of the likelihood of cancer recurrence. U.S. patent application No. 2009/0208514 discloses that the LHX4 gene is a biomarker for screening for a compound for treating or preventing esophageal cancer.

After substantial experiments and persistent research, the applicant has finally conceived a method for evaluating the therapeutic effects of Thalidomide and the application thereof.

### SUMMARY

The present disclosure is directed to a method for evaluating the therapeutic effect of Thalidomide and an application thereof, which evaluates the therapeutic effect of Thalidomide on liver cancer of a patient according to the expression level of the LHX4 gene in the liver of the patient.

In another aspect, the present disclosure discloses a method for evaluating a therapeutic effect of Thalidomide, comprising steps of obtaining tumorous tissue and non-tumorous tissues of a liver from a patient suffering from liver cancer; measuring a first normalized LHX4 expression level in the tumorous tissue and a second normalized LHX4 expression level in the non-tumorous tissue; obtaining a ratio of the first normalized LHX4 expression level to the second normalized LHX4 expression level; and comparing the ratio with a reference value to evaluate the therapeutic effect of Thalidomide on the patient.

In another aspect, the present disclosure discloses a method for evaluating a therapeutic effect of Thalidomide, comprising steps of measuring normalized expression levels of an LHX4 gene in respective tumorous liver tissues from multiple patients; determining a reference expression level based on the respective normalized expression levels of the LHX4 gene; setting a normalized target expression level of the LHX4 gene in a target tumorous liver tissue of a specific patient; and comparing the normalized target expression level with the reference expression level to evaluate the therapeutic effect of Thalidomide on the specific patient.

In another aspect, the present disclosure discloses a method for evaluating a therapeutic effect of Thalidomide, comprising steps of measuring a first LHX4 expression level of in a tumorous liver tissue of a patient; and evaluating the therapeutic effect of Thalidomide on the patient based on the LHX4 expression level.

### BRIEF DESCRIPTION OF DRAWINGS

FIGS. 1A, 1B, 1C, 2A, 2B, 2C, 3A, 3B and 3C are Kaplan-Meier survival curves.

FIG. 4 is a diagram showing a chip that detects LHX4 expression levels in tumorous and non-tumorous liver tissues.

### DETAILED DESCRIPTION

The present disclosure can be fully understood and accomplished by the skilled person according to the following embodiments. However, the practice of present method is not limited to the following embodiments.

Samples of liver tissue were obtained from 48 patients, who suffered HCC at stage II or III (defined by American Joint Committee on Cancer, AJCC) and underwent curative resection for the HCC. All of the patients were enrolled within 4 weeks after surgery. 27 of the patients received Thalidomide 200 mg/day and the others received a placebo 200 mg/day for 12 months or until HCC recurrence.

The systematic name and formula of Thalidomide are (RS)-2-(2,6-dioxopiperidin-3-yl)-1H-isoindole-1,3(2H)-dione and C₁₃H₁₀N₂O₄. In addition, the structure of Thalidomide is:

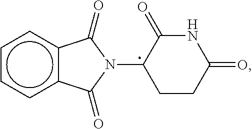

where the symbol “.” indicates the location of an asymmetric carbon atom.

The sample of liver tissue from each patient includes tumorous tissue and non-tumorous tissue neighboring the tumorous tissue. The respective Lhx4 proteins in the tumorous and non-tumorous tissues of each patient were detected using western blot and then quantified by ImageJ software (published by the National Institutes of Health of United States of America). The quantified results of the Lhx4 proteins in the tumorous and non-tumorous tissues were respectively normalized based on the quantified results of β-actin in the tumorous and non-tumorous tissues of the corresponding patient to obtain the (protein) expression levels of the LHX4 gene in the tumorous and non-tumorous tissues of the patient.

For each and every patient, the expression level of the LHX4 gene in the tumorous tissue (called “T” hereinafter) was divided by the expression level of the LHX4 gene in the non-tumorous tissue (called “NT” hereinafter) to obtain a T/NT ratio, which represents the decreasing magnitude of the expression level of the LHX4 gene in the tumorous tissue when compared with the non-tumorous tissue.

In an embodiment, the correlation between the decreasing magnitude of the expression level of the LHX4 gene in the tumorous tissue of a patient and the therapeutic effect of Thalidomide on the liver cancer of the patient is indicated by the HCC recurrence of the patient. Specifically, the end date of observation of a patient's HCC recurrence is set as the date three years after the surgery for the liver cancer.

The patients receiving Thalidomide were divided into two groups, with or without HCC recurrence before the end date. After analysis, the mean decreasing magnitude of the LHX4 expression level of the patients having no HCC recurrence before the end date was more obvious than that of the patients suffering from the HCC recurrence before the end date (the respective mean T/NT ratios were 0.1 (without HCC recurrence) and 0.39 (with HCC recurrence), P=0.024). In contrast, in patients receiving the placebo, the respective mean decreasing magnitudes of the LHX4 expression level of the two groups (divided by suffering from the HCC recurrence before the end date or not) were not significantly different (the respective mean T/NT ratios were 0.29 (without HCC recurrence) and 0.36 (with HCC recurrence), P=0.664). It can be seen that the patients with a lower LHX4 expression level in the tumorous liver tissue had better HCC recurrence-free survival by taking Thalidomide as an adjuvant therapy within three years from undergoing the surgery for the liver cancer.

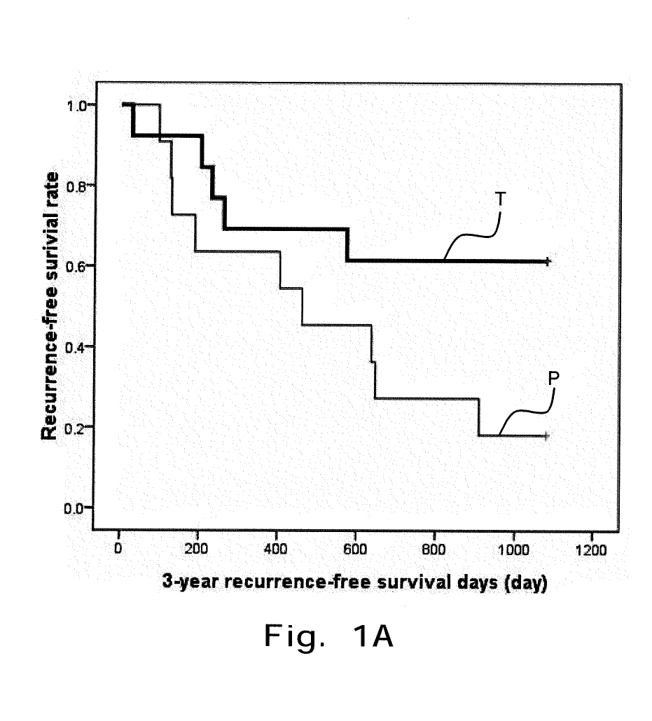

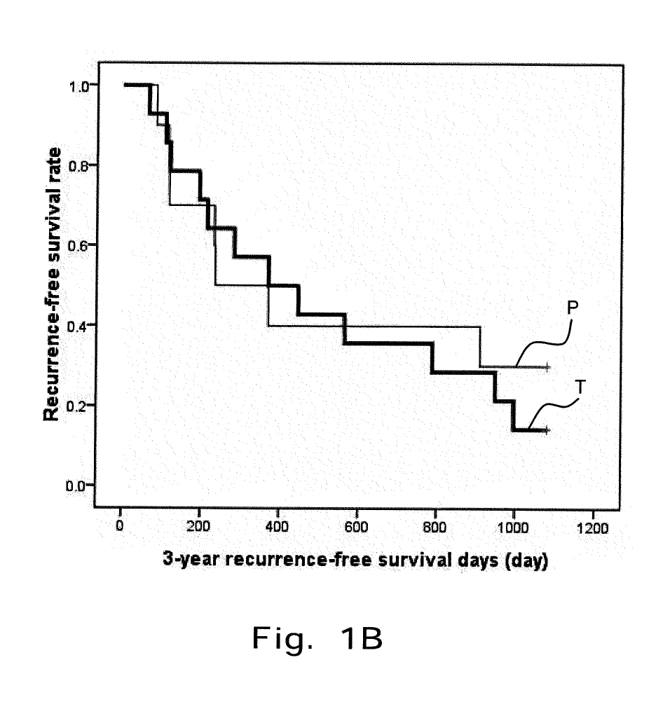

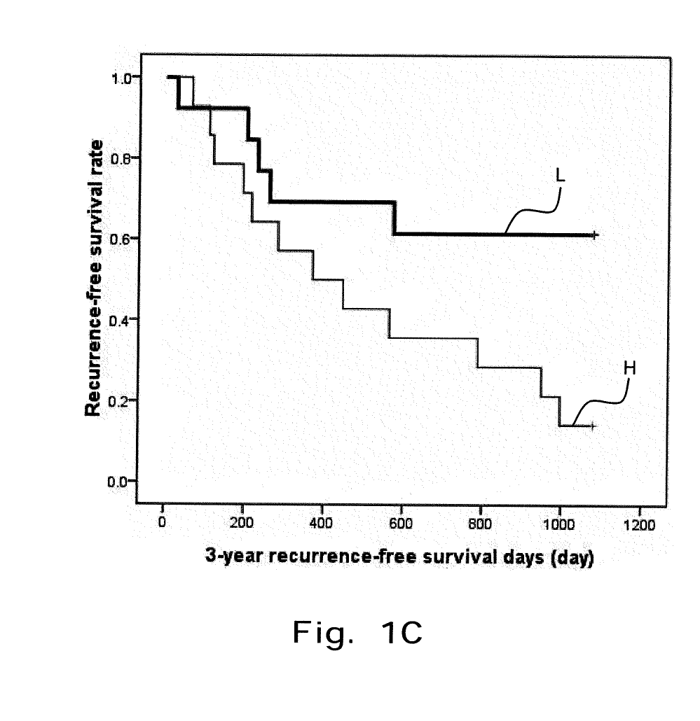

In an embodiment, the 48 patients were divided into two groups according the T/NT ratio to evaluate the therapeutic effect of Thalidomide on the patients. Specifically, if a patient's T/NT ratio was lower than 0.2 (the mean T/NT ratio of the 48 patients), this patient was put in a lower group; and if a patient's T/NT ratio was not lower than 0.2, this patient is in a higher group. The HCC recurrences of the 48 patients were analyzed using Kaplan-Meier survival curves to obtain the 3-year HCC recurrence-free survival days (from the date that the patient underwent the surgery for the liver cancer to the date of observing the HCC recurrence of the patient) for each patient as shown in FIGS. 1A, 1B and 1C, where the event of the Kaplan-Meier survival curve is the first HCC recurrence of a patient after the surgery. In FIG. 1A, for the lower group (24 patients), the mean 3-year HCC recurrence-free survival days for the patients receiving Thalidomide (744 days, 13 patients, shown by curve T) were obviously delayed (P=0.06) when compared with the patients receiving the placebo (526 days, 11 patients, shown by curve P). However, as shown in FIG. 1B, in the higher group (24 patients), the mean 3-year HCC recurrence-free survival days for the patients receiving Thalidomide (521 days, 14 patients, shown by curve T) was not significantly different (P=0.687) from the patients receiving the placebo (535 days, 10 patients, shown by curve P). In addition, as shown in FIG. 1C, in the patients receiving Thalidomide (27 patients), the mean 3-year HCC recurrence-free survival days for the patients in the lower group (744 days, 13 patients, shown by curve L) were obviously delayed (P=0.029) when compared to the patients in the higher group (521 days, 14 patients, shown by curve H).

Table 1 shows the results of when the 48 patients were divided into groups using a T/NT ratio of 0.2, and receiving Thalidomide or the placebo, and the three-year HCC recurrence rates of the patients.

TABLE 1

| | Number of | Number of HCC | HCC recurrence |
|---|---|---|---|
| | total patients | recurrence patients | rate (%) |
| Receiving | 13 | 5 | 38.5 |
| Thalidomide, | | | |
| T/NT ratio < 0.2 | | | |
| Receiving | 11 | 9 | 81.8 |
| placebo, T/NT | | | |
| ratio < 0.2 | | | |
| Receiving | 14 | 12 | 85.7 |
| Thalidomide, | | | |
| T/NT ratio ≧ 0.2 | | | |
| Receiving | 10 | 7 | 70 |
| placebo, T/NT | | | |
| ratio ≧ 0.2 | | | |

As shown in Table 1, it can be seen that the patients who received Thalidomide and whose T/NT ratios were lower than 0.2 have a lower three-year HCC recurrence rate than other groups of patients. That is, for a patient having a relatively low T/NT ratio, the therapeutic effect of Thalidomide obviously decreased the HCC recurrence for the patient.

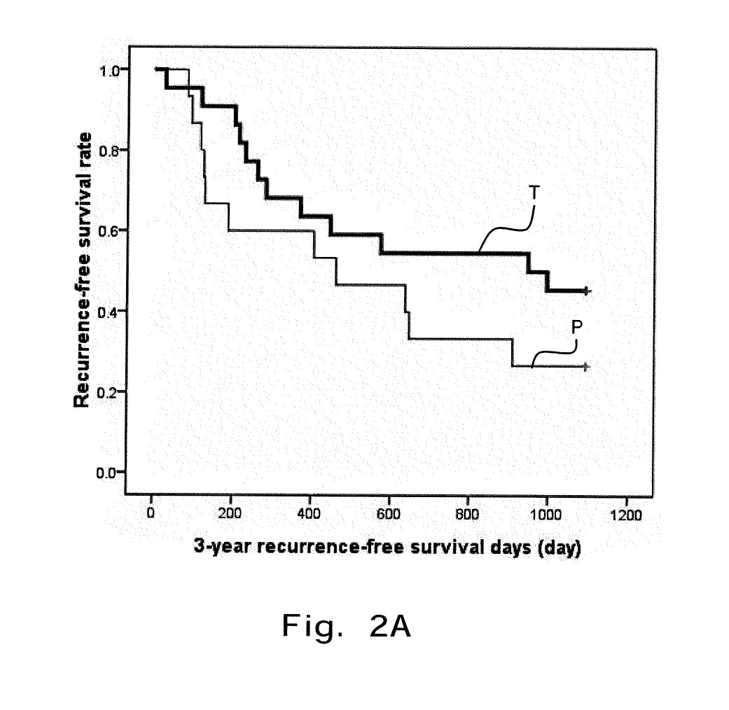

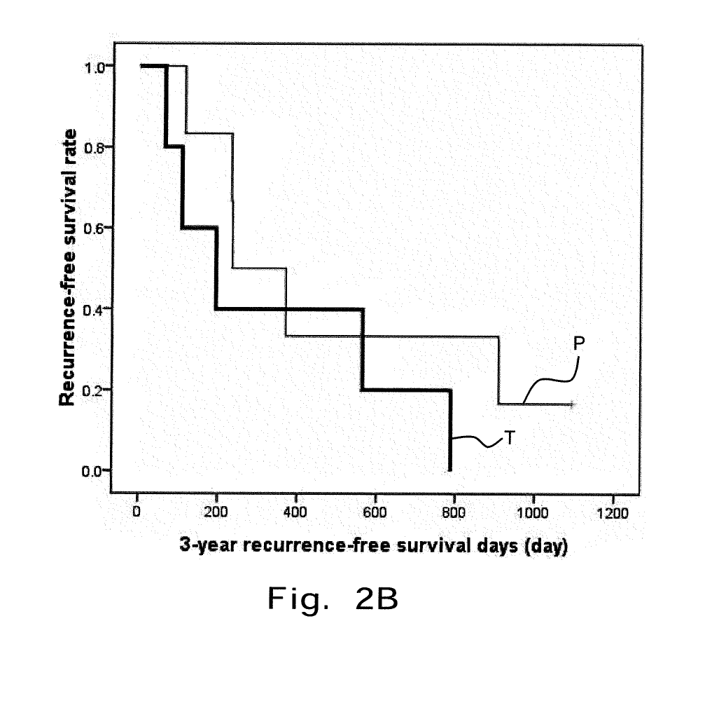

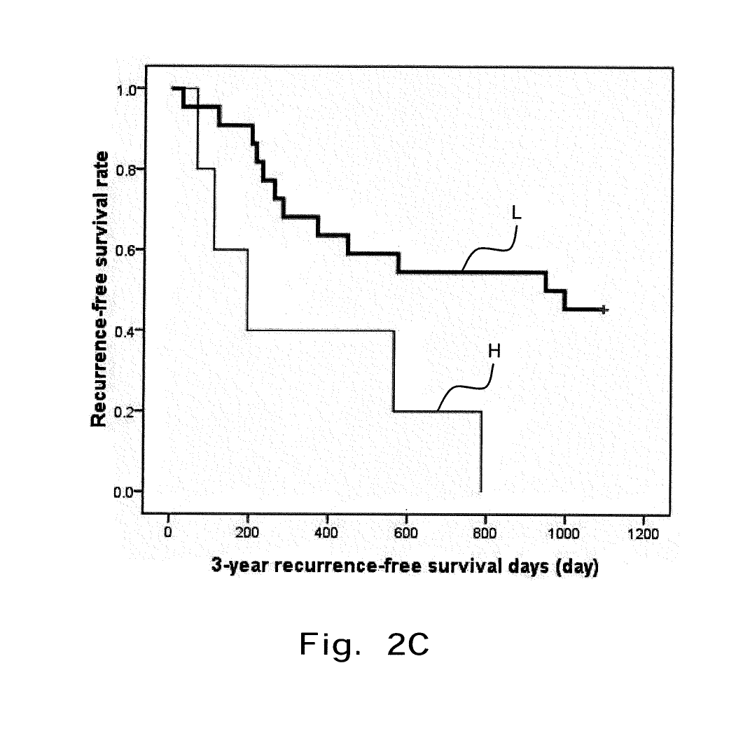

In an embodiment, the 48 patients were divided into a lower group (where the patients had a T/NT ratio lower than 0.5) and a higher group (where the patients had a T/NT ratio not lower than 0.5), and the HCC recurrences of all the patients were analyzed using Kaplan-Meier survival curves as shown in FIGS. 2A, 2B and 2C. Each of FIGS. 2A, 2B and 2C show the respective 3-year HCC recurrence-free survival days (from the date that the patient underwent the surgery for the liver cancer to the date of observing the HCC recurrence of the patient) of the patients, where the event in the curves is the first HCC recurrence of a patient after the surgery. In FIG. 2A, in the lower group (37 patients), the mean 3-year HCC recurrence-free survival days of the patients receiving Thalidomide (711 days, 22 patients, shown by curve T) were obviously delayed (P=0.2) when compared to the patients receiving the placebo (545 days, 15 patients, shown by curve P). However, as shown in FIG. 2B, in the higher group (11 patients), the mean 3-year HCC recurrence-free survival days of the patients receiving Thalidomide (344 days, 5 patients, shown by curve T) was not significantly different (P=0.272) from that of the patients receiving the placebo (493 days, 6 patients, shown by curve P). In addition, as shown in FIG. 2C, in the patients receiving Thalidomide (27 patients), the mean 3-year HCC recurrence-free survival days of the patients in the lower group (711 days, 22 patients, shown by curve L) were obviously delayed (P=0.011) when compared with the patients in the higher group (344 days, 5 patients, shown by curve H).

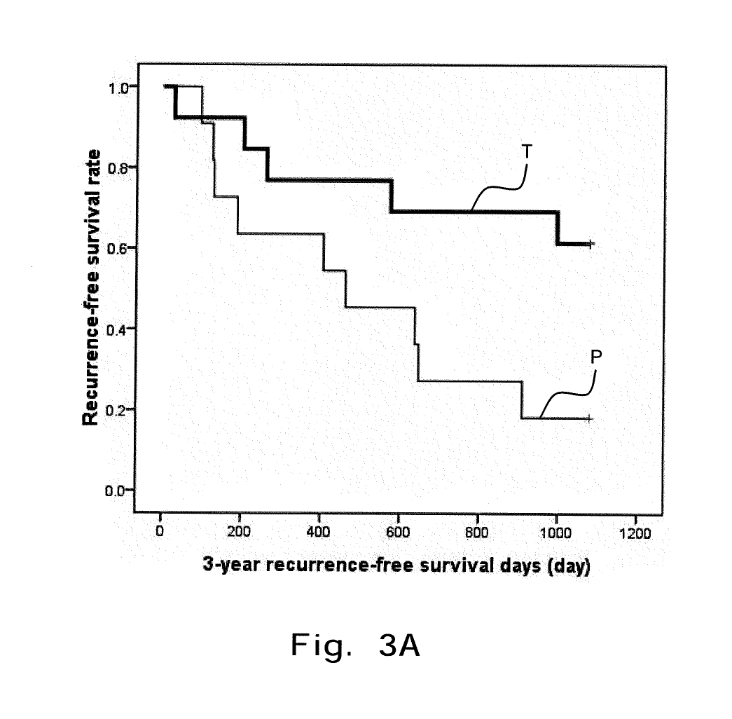

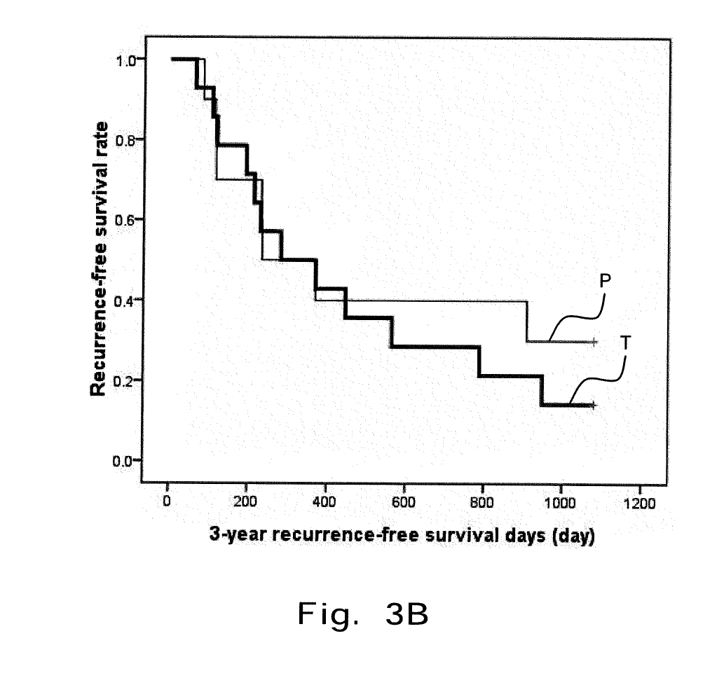

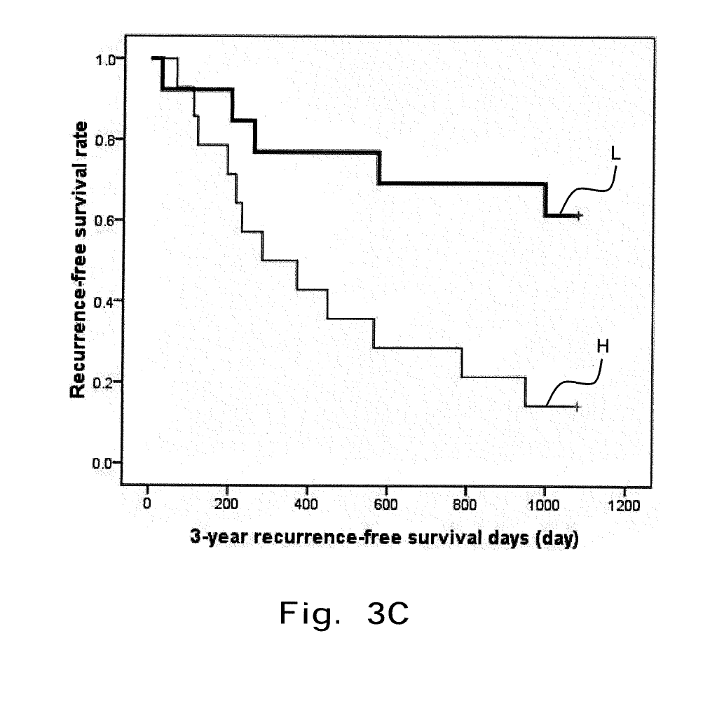

In an embodiment, for each of the 48 patients, the LHX4 expression level in the tumorous tissue was obtained through normalizing the quantified result of the western blot of the Lhx4 protein of the tumorous tissue based on a specific amount of β-actin in the tumorous tissue. After the normalization, the LHX4 expression levels in the tumorous tissue of all the patients, normalized by the same amount of β-actin, were obtained. Subsequently, the 48 patients were divided into a lower group (where the patients had an LHX4 expression level in the tumorous tissue lower than 0.17, which was the median of the LHX4 expression levels in the tumorous liver tissue of the 48 patients) and a higher group (where the patients had an LHX4 expression level in the tumorous tissue not lower than 0.17), and the HCC recurrences of all the patients were analyzed using Kaplan-Meier survival curves as shown in FIGS. 3A, 3B and 3C. Each of FIGS. 3A, 3B and 3C show the respective 3-year HCC recurrence-free survival days (from the date that the patient underwent the surgery for the liver cancer to the date of observing the HCC recurrence of the patient) of the patients, where the event of the curves is the first HCC recurrence in a patient after the surgery. In FIG. 3A, in the lower group (24 patients), the mean 3-year HCC recurrence-free survival days of the patients receiving Thalidomide (833 days, 13 patients, shown by curve T) were obviously delayed (P=0.033) when compared with the patients receiving the placebo (526 days, 11 patients, shown by curve P). However, as shown in FIG. 3B, in the higher group (24 patients), the mean 3-year HCC recurrence-free survival days of the patients receiving Thalidomide (466 days, 14 patients, shown by curve T) was not significantly different (P=0.567) from that of the patients receiving the placebo (535 days, 10 patients, shown by curve P). In addition, as shown in FIG. 3C, in the patients receiving Thalidomide (27 patients), the mean 3-year HCC recurrence-free survival days of the patients in the lower group (833 days, 13 patients, shown by curve L) were obviously delayed (P=0.011) when compared with the patients in the higher group (466 days, 14 patients, shown by curve H).

In an embodiment, the 48 patients were divided into a lower group (where the patients had an LHX4 expression level in the tumorous tissue lower than the arithmetic average of the respective LHX4 expression levels in the tumorous liver tissue of the 48 patients) and a higher group (where the patients had an LHX4 expression level in the tumorous tissue not lower than the arithmetic average), where the LHX4 expression level in the tumorous tissue of each patient was obtained through normalization based on the same amount of β-actin in the patient's tumorous tissue as mentioned above. In this embodiment, in the patients receiving Thalidomide, the mean 3-year HCC recurrence-free survival days of the patients in the lower group were obviously delayed when compared with that of the patients in the higher group.

Based on at least the above embodiments, it can be seen that if a patient's LHX4 expression level in the tumorous tissue or T/NT ratio is relatively low, the therapeutic effect of Thalidomide is relatively good on this patient, and can obviously delay the HCC recurrence (date) or decrease the HCC recurrence of this patient after this patient receives the curative treatment against the liver cancer. In contrast, if a patient's LHX4 expression level in the tumorous tissue or T/NT ratio is relatively high, the therapeutic effect of Thalidomide is relatively poor. Clinically, the curative treatment against the liver cancer includes at least liver transplantation, tumor resection and tumor ablation.

In an embodiment, the administration of Thalidomide to a patient who received the curative treatment against the liver cancer was 50 mg to 200 mg daily and more than six months from the date that the patient received the curative treatment.

In an embodiment, the LHX4 expression levels of the tumorous and non-tumorous liver tissues were detected and measured through immunohistochemistry (IHC) of sections of the tumorous and non-tumorous liver tissues.

In an embodiment, the LHX4 expression levels of the tumorous and non-tumorous liver tissues were detected and measured through Quantitative Real-Time RT-PCR (qRT-PCR) using mRNAs obtained from the tumorous and non-tumorous liver tissues as materials.

In an embodiment, the therapeutic effect of Thalidomide on a specific patient suffering HCC was evaluated based on a reference value obtained from LHX4 expression levels in tumorous tissue of multiple patients if the specific patient receives a curative treatment against the HCC. Specifically, the reference value was determined according to the LHX4 expression levels in the tumorous tissue of the multiple patients, and then the LHX4 expression level in the tumorous tissue of the specific patient (defined as an evaluative value) was compared with the reference value. After the comparison, if the evaluative value is higher than the reference value, the therapeutic effect of Thalidomide is relatively poor on the specific patient; and if the evaluative value is not higher than the reference value, the therapeutic effect of Thalidomide is relatively good on the specific patient, and can obviously delay the HCC recurrence (date) or decrease the HCC recurrence rate of the specific patient after the specific patient receives the curative treatment against the HCC.

The reference value can be, but not limited to, an arithmetic average or a median of the LHX4 expression levels of the multiple patients. In another embodiment, the LHX4 expression levels in the tumorous tissue of the multiple patients were screened to remove extreme value(s), such as a value over a specific percentage or threshold, and the reference value was determined based on the remaining LHX4 expression levels.

In an embodiment, the population of multiple patients includes the specific patient, and therefore the LHX4 expression level in tumorous tissue of the specific patient is one of the LHX4 expression levels in the tumorous tissue of the multiple patients. In another embodiment, the population of multiple patients does not include the specific patient.

In an embodiment, after the comparison between the evaluative and the reference values, the LHX4 expression level in tumorous tissue of the specific patient (the evaluative value) was incorporated into a collection of those of the multiple patients so as to generate a new collection including the LHX4 expression levels in the tumorous tissue of the specific and multiple patients and to obtain an update reference value based on the new collection, where the update reference will be more presentative than the reference value. In this embodiment, the population of multiple patients does not include the specific patient. Then the update reference value can be used to compare with an LHX4 expression level in tumorous tissue (a second evaluative value) of a second specific patient to evaluate the therapeutic effect of Thalidomide on the second specific patient.

In an embodiment, the above-mentioned reference value was obtained from the T/NT ratios of the multiple patients rather than the LHX4 expression levels in the tumorous tissue of the multiple patients, and the evaluative value is the T/NT ratio of the specific patient rather than the LHX4 expression level in the tumorous tissue of the specific patient. In this embodiment, the T/NT ratio of the subsequent patient was used to compare with the update reference value to evaluate the therapeutic effect of Thalidomide on the subsequent patient.

The above-mentioned embodiments regarding evaluating the therapeutic effect of Thalidomide on the patient based on the (update) reference and the (second) evaluative values are computer-implementable processes, and the parameters used in the processes, such as the respective LHX4 expression levels in the tumorous tissue and the T/NT ratios of the (second) specific and multiple patients and the (update) reference and the (second) evaluative values, are stored in a memory of the implementing computer.

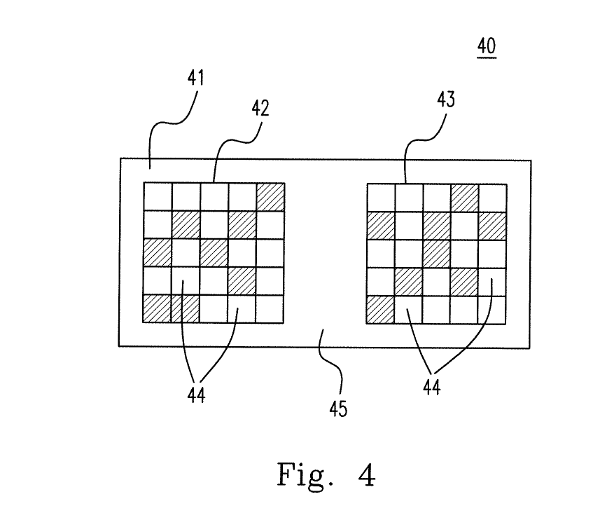

Please refer to FIG. 4, which shows a device used to evaluate the therapeutic effect of Thalidomide. In FIG. 4, a chip 40 includes a substrate 41, a first area 42, a second area 43 and indicative material 44, where the first and the second areas 42, 43 are located on the substrate 41 and used to respectively support tumorous and non-tumorous liver tissue samples from a patient, and the indicative material 44 is distributed on both the first and the second areas 42, 43, and used to specifically bind to at least one of mRNA of the LHX4 and the Lhx4 protein. Through the indicative material 44, the LHX4 expression levels in the tumorous and non-tumorous liver tissue samples are detected and revealed, and therefore the LHX4 expression level in the tumorous liver tissue and the T/NT ratio of the patient can be obtained. In addition, there is a gap area 45 configured on the chip 40 between the first and the second areas 42, 43 to prevent the tumorous and non-tumorous liver tissue samples from touching each other. When the indicative material 44 can specifically bind to the mRNA of LHX4, the tumorous and non-tumorous liver tissue samples on the first and the second areas 42, 43 are total RNAs in the tumorous and non-tumorous liver tissues. When the indicative material 44 can specifically bind to the Lhx4 protein, the tumorous and non-tumorous liver tissue samples on the first and the second areas 42, 43 are tumorous and non-tumorous liver tissue lysates.

In an embodiment, the LHX4 expression levels revealed on the first and the second areas 42, 43 are measured by a machine such as a spectrophotometer detecting and measuring the fluorescence emitted by the indicative material 44, so as to obtain the LHX4 expression level in the tumorous liver tissue and the T/NT ratio of the patient.

In an embodiment, the first and the second areas 42, 43 having the indicative material 44 are configured on separate chips so as to form a system to evaluate the therapeutic effect of Thalidomide.

### EMBODIMENTS

Embodiment 1 is a method for evaluating a therapeutic effect of Thalidomide, comprising steps of obtaining tumorous tissue and non-tumorous tissues of a liver from a patient suffering from liver cancer, measuring a first normalized LHX4 expression level in the tumorous tissue and a second normalized LHX4 expression level in the non-tumorous tissue, obtaining a ratio of the first normalized LHX4 expression level to the second normalized LHX4 expression level, and comparing the ratio with a reference value to evaluate the therapeutic effect of Thalidomide on the patient.

Embodiment 2 is a method of Embodiment 1, where the therapeutic effect of Thalidomide is to delay a tumor recurrence in the liver of the patient.

Embodiment 3 is a method of either of Embodiments 1 and 2, where the therapeutic effect of Thalidomide is to delay the tumor recurrence in the liver of the patient after the patient receives a curative treatment against the liver cancer.

Embodiment 4 is a method of Embodiment 2 or 3, where if the ratio is higher than the reference value, the therapeutic effect of Thalidomide is relatively poor, and if the ratio is not higher than the reference value, the therapeutic effect of Thalidomide is relatively good.

Embodiment 5 is a method of any of Embodiments 1 to 4, where the reference value ranges between 0.2 and 0.5, inclusive.

Embodiment 6 is a method of any of Embodiments 1 to 5, where the expression levels are determined with regard to protein levels.

Embodiment 7 is a method of any of Embodiments 1 to 5, where the expression levels are determined with regard to mRNA levels.

Embodiment 8 is a method for evaluating a therapeutic effect of Thalidomide, comprising steps of measuring respective normalized expression levels of an LHX4 gene in respective tumorous liver tissues from multiple patients, determining a reference expression level based on the respective normalized expression levels of the LHX4 gene, setting a normalized target expression level of the LHX4 gene in a target tumorous liver tissue of a specific patient, and comparing the normalized target expression level with the reference expression level to evaluate the therapeutic effect of Thalidomide on the specific patient.

Embodiment 9 is a method of Embodiment 8, where the therapeutic effect of Thalidomide is to delay a tumor recurrence in a liver of the specific patient.

Embodiment 10 is a method of Embodiment 8 or 9, where the therapeutic effect of Thalidomide is to delay the tumor recurrence in the liver of the specific patient after the specific patient receives a curative treatment against the liver cancer.

Embodiment 11 is a method of any of Embodiments 8 to 10, where if the normalized target expression level is higher than the reference expression level, the therapeutic effect of Thalidomide is relatively poor, and if the normalized target expression level is not higher than the reference expression level, the therapeutic effect of Thalidomide is relatively good.

Embodiment 12 is a method of any of Embodiments 8 to 11, where the reference expression level is one of an arithmetic average of the respective normalized expression levels of the LHX4 gene and a median of the respective normalized expression levels of the LHX4 gene.

Embodiment 13 is a method of any of Embodiments 8 to 12, further comprising steps of determining an updated reference expression level based on the respective normalized and the normalized target expression levels of the LHX4 gene, setting a second normalized target expression level of the LHX4 gene in tumorous liver tissue of a second specific patient, and comparing the second target normalized expression level with the updated reference expression level to evaluate the therapeutic effect of Thalidomide on the second specific patient.

Embodiment 14 is a method of any of Embodiments 8 to 13, where the method is implemented by a computer having a memory, and the respective normalized and the normalized target expression levels of the LHX4 gene are stored in the memory.

Embodiment 15 is a method of any of Embodiments 8 to 14, where the population of multiple patients includes the specific patient.

Embodiment 16 is a method of any of Embodiments 8 to 14, where the population of multiple patients does not include the specific patient.

Embodiment 17 is a method for evaluating a therapeutic effect of Thalidomide, comprising steps of measuring a first LHX4 expression level in a tumorous liver tissue of a patient, and evaluating the therapeutic effect of Thalidomide on the patient based on the LHX4 expression level.

Embodiment 18 is a method of Embodiment 18, further comprising steps of measuring a second LHX4 expression level in a non-tumorous liver tissue of the patient, deriving an evaluative value based on the first and the second LHX4 expression levels, determining a reference value, and comparing the evaluative value with the reference value to evaluate the therapeutic effect of Thalidomide on the patient.

Embodiment 19 is a method of either of Embodiments 17 and 18, where the evaluative value is a ratio of the first expression level to the second expression level, wherein if the ratio is higher than the reference value, the therapeutic effect of Thalidomide is relatively poor, and if the ratio is not higher than the reference value, the therapeutic effect of Thalidomide is relatively good.

Embodiment 20 is a method of any of Embodiments 17 to 19, where the therapeutic effect of Thalidomide is at least one of delaying a tumor recurrence in a liver of the patient and decreasing a tumor recurrence rate of a liver cancer of the patient after the patient receives a curative treatment against the liver cancer.

While this disclosure is of terms of what is presently considered to be the most practical and preferred embodiments, it is to be understood that the disclosure is not limited to the disclosed embodiments. Therefore, it is intended to cover various modifications and similar arrangements included within the spirit and scope of the appended claims, which are to be accorded with the broadest interpretation so as to encompass all such modifications and similar structures.

What is claimed is:

1. A method for evaluating a therapeutic effect of Thalidomide, comprising steps of:
obtaining tumorous tissue and non-tumorous tissues of a liver from a patient suffering from a liver cancer;
measuring a first normalized LHX4 expression level in the tumorous tissue and a second normalized LHX4 expression level in the non-tumorous tissue;
obtaining a ratio of the first normalized LHX4 expression level to the second normalized LHX4 expression level; and
comparing the ratio with a reference value to evaluate the therapeutic effect of Thalidomide on the patient.

2. The method as claimed in claim 1, wherein the therapeutic effect of Thalidomide is to delay a tumor recurrence in the liver of the patient.

3. The method as claimed in claim 2, wherein the therapeutic effect of Thalidomide is to delay the tumor recurrence in the liver of the patient after the patient receives a curative treatment against the liver cancer.

4. The method as claimed in claim 1, wherein if the ratio is higher than the reference value, the therapeutic effect of Thalidomide is relatively poor, and if the ratio is not higher than the reference value, the therapeutic effect of Thalidomide is relatively good.

5. The method as claimed in claim 1, wherein the reference value ranges between 0.2 and 0.5, inclusive.

6. The method as claimed in claim 1, wherein the expression levels are determined with regard to protein levels.

7. The method as claimed in claim 1, wherein the expression levels are determined with regard to mRNA levels.

8. A method for evaluating a therapeutic effect of Thalidomide, comprising steps of:
measuring respective normalized expression levels of an LHX4 gene in respective tumorous liver tissues from multiple patients;
determining a reference expression level based on the respective normalized expression levels of the LHX4 gene;
setting a normalized target expression level of the LHX4 gene in a target tumorous liver tissue of a specific patient; and
comparing the normalized target expression level with the reference expression level to evaluate the therapeutic effect of Thalidomide on the specific patient.

9. The method as claimed in claim 8, wherein the therapeutic effect of Thalidomide is to delay a tumor recurrence in a liver of the specific patient.

10. The method as claimed in claim 9, wherein the therapeutic effect of Thalidomide is to delay the tumor recurrence in the liver of the specific patient after the specific patient receives a curative treatment against the liver cancer.

11. The method as claimed in claim 8, wherein if the normalized target expression level is higher than the reference expression level, the therapeutic effect of Thalidomide is relatively poor, and if the normalized target expression level is not higher than the reference expression level, the therapeutic effect of Thalidomide is relatively good.

12. The method as claimed in claim 8, wherein the reference expression level is one of an arithmetic average of the respective normalized expression levels of the LHX4 gene and a median of the respective normalized expression levels of the LHX4 gene.

13. The method as claimed in claim 8, wherein the method is implemented by a computer having a memory, and the respective normalized expression levels of the LHX4 gene are stored in the memory.

14. The method as claimed in claim 8, further comprising steps of:
determining an updated reference expression level based on the respective normalized and the normalized target expression levels of the LHX4 gene;
setting a second normalized target expression level of the LHX4 gene in tumorous liver tissue of a second specific patient; and
comparing the second target normalized expression level with the updated reference expression level to evaluate the therapeutic effect of Thalidomide on the second specific patient.

15. The method as claimed in claim 14, wherein the method is implemented by a computer having a memory, and the respective normalized and the normalized target expression levels of the LHX4 gene are stored in the memory.

16. The method as claimed in claim 8, wherein the population of multiple patients includes the specific patient.

17. The method as claimed in claim 8, wherein the population of multiple patients does not include the specific patient.

18. A method for evaluating a therapeutic effect of Thalidomide, comprising steps of:
measuring a first LHX4 expression level of in a tumorous liver tissue of a patient; and
evaluating the therapeutic effect of Thalidomide on the patient based on the LHX4 expression level.

19. The method as claimed in claim 18, further comprising steps of:
measuring a second LHX4 expression level in a non-tumorous liver tissue of the patient;
deriving an evaluative value based on the first and the second LHX4 expression levels;
determining a reference value; and
comparing the evaluative value with the reference value to evaluate the therapeutic effect of Thalidomide on the patient.

20. The method as claimed in claim 18, wherein the therapeutic effect of Thalidomide is at least one of delaying a tumor recurrence in a liver of the patient and decreasing a tumor recurrence rate of a liver cancer of the patient after the patient receives a curative treatment against the liver cancer.
